(19) United States
(12) Patent Application Publication
(10) Pub. No.: US 2019/0125751 A1
(43) Pub. Date: May 2, 2019

(54) ANTICANCER COMBINATION THERAPY

(71) Applicant: BOEHRINGER INGELHEIM INTERNATIONAL GMBH, Ingelheim am Rhein (DE)

(72) Inventors: Flavio SOLCA, Vienna (AT); Mahmoud OULD KACI, Ridgefield, CT (US); Mark PETRONCZKI, Vienna (AT); Ulrike TONTSCH-GRUNT, Baden (AT); Victoria ZAZULINA, Camberley (GB)

(21) Appl. No.: 16/301,463

(22) PCT Filed: May 15, 2017
(86) PCT No.: PCT/EP2017/061585

§ 371 Date: Nov. 14, 2018

(30) Foreign Application Priority Data
May 18, 2016 (EP) 16170179.2

(51) Int. Cl. A61K 31/519; A61K 31/517; A61P 35/00

(52) U.S. Cl. CPC A61K 31/519; A61P 35/00; A61K 31/517

(57) ABSTRACT

The invention describes anti-cancer therapies comprising using a 3G-EGFR inhibitor and an irreversible (2^{nd} generation) EGFR TKI, each as described herein.

Epidermal growth factor receptor (EGFR) tyrosine kinase inhibitors (TKI) have marked a new era in the treatment of advanced non-small cell lung cancer (NSCLC). Over the last decade, EGFR TKIs established a remarkable therapeutic benefit in the patients with advanced NSCLC harboring EGFR activating mutations [1-7]. Unfortunately, however, efficacy of 1^{st} generation EGFR TKIs gefitinib and erlotinib is ultimately limited by inevitable development of acquired resistance (AR) after median of 10 to 12 months [8-11]. T790M is known to be the most common mechanism of AR observed in approximately 50 to 60% of patients. In this gatekeeper mutation, a well conserved threonine at codon 790 in exon 20 of EGFR undergoes substitution to bulkier methionine, which leads to steric hindrance of erlotinib binding in the ATP-kinase-binding pocket [8]. 2^{nd} generation EGFR TKIs, including afatinib (BIBW2992) and dacomitinib (PF299804), effectively inhibit T790M-containing cell lines in several preclinical models. In addition, mutant selective, 3^{rd} generation EGFR TKI, which comprises the irreversible pyrimidine-based WZ 4002 and newer compounds, i.e. AZD9291, CO1686, and HM61713 (BI 1482694) [12], have been developed. Strikingly, recent preclinical and preliminary clinical data demonstrated an outstanding clinical efficacy of 3^{rd} generation EGFR TKIs in patients with advanced NSCLC harboring T790M [13-18]. However, despite 3^{rd} generation EGFR TKIs emerging at the forefront in the treatment of EGFR mutant NSCLC, in practice patients finally experience disease progression regardless of clinical responses. It suggests the successive evolvement of acquired resistance beyond T790M, that is, 3^{rd} generation EGFR TKIs alone, are insufficient to control the disease.

Although, little is known about the various mechanisms of resistance to 3^{rd} generation EGFR TKIs, recent studies have identified the acquired EGFR C797S mutation as resistance mechanism. The investigators of the AZD9291 clinical trials have shown that biological mechanisms of resistance to this drug can be readily identified in cell-free plasma DNA from patients. The most frequent mechanism identified (40% of 15 EGFR-T790M cases treated with AZD9291) was the acquisition of the EGFR-C797S mutation in exon 20 of EGFR. These investigators and others show in preclinical models that EGFR-exon 19 deletion+T790M+C797S and EGFR−L858R+T790M+C797S generate proteins that are resistant to AZD9291, rociletinib and all irreversible EGFR TKIs (including quinazolone- and pyrimidine-based compounds) by impairing covalent binding of these drugs to the C797 amino-acid residue of EGFR [19-21].

Hence there is still a need for additional treatment options for patients with cancer and, in particular, solid tumors. There is also a need for additional treatment options for patients with lung cancer, such as NSCLC. Designing a combination treatment strategy that can prevent or suppress double or triple-mutant EGFR resistance mechanisms is warranted.

It is thus an object of the invention to provide combination treatments/methods of combination treatment providing certain advantages compared to treatments/methods of treatment currently used and/or known in the prior art. These advantages may include in vivo efficacy (e.g. improved clinical response, extend of the response, increase of the rate of response, duration of response, response rate, disease stabilization rate, duration of stabilization, time to disease progression, progression free survival (PFS) and/or overall survival (OS), later occurrence of resistance and the like), safe and well tolerated administration and reduced frequency and severity of adverse events, in particular reduced frequency and severity of the typical EGFR-mediated adverse events.

In this context, the inventors of the current application, surprisingly, discovered that the use of a mutant-selective 3^{rd} generation EGFR TKI (preferably HM61713 (=BI 1482694=olmutinib)) in combination with an irreversible (2^{nd} generation) EGFR TKI (preferably afatinib) has the potential to improve clinical outcome compared to the use of either an irreversible (2^{nd} generation) EGFR TKI alone or a mutant-selective 3^{rd} generation EGFR TKI alone.

Thus, the invention relates to methods for the treatment and/or prevention of oncological or hyperproliferative diseases, in particular cancer, comprising the combined administration of a mutant-selective 3^{rd} generation EGFR TKI (referred to herein as “3G-EGFR inhibitor”) and an irreversible (2^{nd} generation) EGFR TKI, as well as to medical uses, to uses, to pharmaceutical compositions or combinations and kits comprising such active ingredients.

Further, the invention relates to anti-cancer therapies comprising using a 3G-EGFR inhibitor and an irreversible (2^{nd} generation) EGFR TKI, each as described herein, in combination.

For the treatment of diseases of oncological nature, a large number of anticancer agents (including target-specific and non-target-specific anticancer agents) have already been suggested, which can be used as monotherapy or as combination therapy involving more than one agent (e.g. dual or triple combination therapy) and/or which may be combined with radiotherapy (e.g. irradiation treatment), radio-immunotherapy and/or surgery.

Even if the concept of combining several therapeutic agents or therapies has already been suggested, and although various combination therapies are under investigation and in clinical trials, there is still a need for new and efficient therapies of cancer diseases, which show advantages over standard therapies, such as for example better treatment outcome, beneficial effects, superior efficacy and/or improved tolerability, such as e.g. reduced side effects of the combined treatment.

It is a purpose of the present invention to provide combination therapies with the active agents described herein for treating or controlling various malignancies (e.g. based on cooperative, complementary, interactive or improving effects of the active components involved in combination).

Thus, in one aspect, the invention provides a method of treating and/or preventing an oncological or hyperproliferative disease, in particular cancer, comprising administering to a patient in need thereof a therapeutically effective amount of a 3G-EGFR inhibitor and a therapeutically effective amount of an irreversible (2^{nd} generation) EGFR TKI, each as described herein.

Such a combined treatment may be given as a non-fixed (e.g. free) combination of the substances or in the form of a fixed combination, including kit-of-parts.

In another aspect, the invention refers to a combination of a 3G-EGFR inhibitor and an irreversible (2^{nd} generation) EGFR TKI, each as described herein, particularly for use in a method of treating and/or preventing an oncological or hyperproliferative disease, in particular a cancer disease e.g. as described herein, said method comprising administering to a patient in need thereof a therapeutically effective amount of the combination.

In another aspect, the invention refers to a 3G-EGFR inhibitor as described herein for use in a method of treating and/or preventing an oncological or hyperproliferative disease, in particular cancer, said method comprising administering the 3G-EGFR inhibitor in combination with an irreversible (2^{nd} generation) EGFR TKI as described herein to a patient in need thereof.

In another aspect, the invention refers to an irreversible (2^{nd} generation) EGFR TKI as described herein for use in a method of treating and/or preventing an oncological or hyperproliferative disease, in particular cancer, said method comprising administering the irreversible (2^{nd} generation) EGFR TKI in combination with a 3G-EGFR inhibitor as described herein to a patient in need thereof.

In another aspect, the invention refers to a kit comprising
a first pharmaceutical composition or dosage form comprising a 3G-EGFR inhibitor as described herein, and, optionally one or more pharmaceutically acceptable carriers, excipients and/or vehicles, and
a second pharmaceutical composition or dosage form comprising an irreversible (2^{nd} generation) EGFR TKI as described herein, and,
optionally one or more pharmaceutically acceptable carriers, excipients and/or vehicles.

In another aspect, the invention refers to the aforementioned kit further comprising
a package insert comprising printed instructions for simultaneous, concurrent, sequential, successive, alternate or separate use in the treatment and/or prevention of an oncological or hyperproliferative disease, in particular cancer, in a patient in need thereof.

In another aspect, the invention refers to the aforementioned kits for use in a method of treating and/or preventing an oncological or hyperproliferative disease, in particular cancer.

In another aspect, the invention refers to a pharmaceutical composition comprising
a 3G-EGFR inhibitor as described herein,
an irreversible (2^{nd} generation) EGFR TKI as described herein, and,
optionally, one or more pharmaceutically acceptable carriers, excipients and/or vehicles.

In another aspect, the invention refers to the use of a 3G-EGFR inhibitor as described herein for preparing a pharmaceutical composition for treating and/or preventing an oncological or hyperproliferative disease, in particular cancer (such as e.g. a cancer disease as described herein), wherein the 3G-EGFR inhibitor is to be used in combination with an irreversible (2^{nd} generation) EGFR TKI as described herein.

In another aspect, the invention refers to the use of an irreversible (2^{nd} generation) EGFR TKI as described herein for preparing a pharmaceutical composition for treating and/or preventing an oncological or hyperproliferative disease, in particular cancer (such as e.g. a cancer disease as described herein), wherein the irreversible (2^{nd} generation) EGFR TKI is to be used in combination with a 3G-EGFR inhibitor as described herein.

In another aspect, the invention refers to the use of a 3G-EGFR inhibitor and an irreversible (2^{nd} generation) EGFR TKI, each as described herein, for preparing a pharmaceutical composition for treating and/or preventing an oncological or hyperproliferative disease, in particular cancer (such as e.g. a cancer disease as described herein).

In another aspect, the invention refers to a combination, composition or kit according to the invention comprising, consisting or consisting essentially of a 3G-EGFR inhibitor and an irreversible (2^{nd} generation) EGFR TKI, each as described herein, e.g. for treating and/or preventing an oncological or hyperproliferative disease, in particular cancer (e.g. a cancer disease as described herein).

In another aspect, the invention refers to a combination, composition or kit according to the invention optionally further comprising one or more other therapeutic agents.

In another aspect, the invention refers to a method or a 3G-EGFR inhibitor for use or an irreversible (2^{nd} generation) EGFR TKI for use or use or pharmaceutical composition for use or kit for use according to the invention optionally further comprising administering or involving one or more other therapeutic agents.

### 3G-EGFR Inhibitor

The 3G-EGFR inhibitor within the meaning of this invention is a compound which selectively inhibits EGFR mutant isoforms while sparing to some extent wild type EGFR.

Preferably, this inhibition is irreversible.

Preferably, the 3G-EGFR inhibitor within this invention is N-(3-{2-[4-(4-methyl-piperazin-1-yl)-phenylamino]-thieno[3,2-d]pyrimidin-4-yloxy}-phenyl)-acrylamide (compound A, also known as BI 1482694 and HM 61713 and olmutinib). The term “3G-EGFR inhibitor” as used herein also includes compound A in the form of a tautomer, of a pharmaceutically acceptable salt, of a hydrate or of a solvate. It also includes compound A in all its solid, preferably crystalline, forms and in all the crystalline forms of its pharmaceutically acceptable salts, hydrates and solvates.

### Compound A: N-(3-{2-[4-(4-methyl-piperazin-1-yl)-phenylamino]-thieno[3,2-d]pyrimidin-4-yloxy}-phenyl)-acrylamide

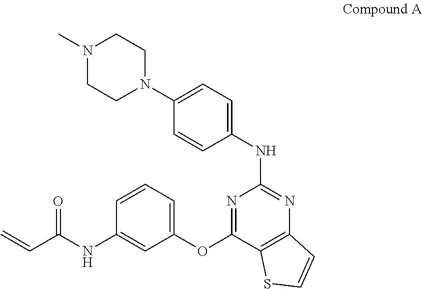 [SMILES: C=CC(=O)Nc1cccc(Oc2nc(Nc3ccc(N4CCN(C)CC4)cc3)nc3ccsc23)c1]

Compound A, its synthesis and properties are disclosed in WO 2011/162515 which is incorporated by reference in its entirety (example compound 1, page 33).

Compound A is a small molecule epidermal growth factor receptor (EGFR) mutant-specific inhibitor. It is being evaluated as a novel oral therapy for the treatment of non-small cell lung cancer (NSCLC) with EGFR mutations, including EGFR T790M (associated with acquired resistance to currently approved EGFR-targeting agents gefitinib, erlotinib, afatinib) and mutations conferring sensitivity to EGFR tyrosine-kinase inhibitors (including EGFR Del19, EGFR L858R etc.). In vitro data confirms that compound A is an irreversible EGFR mutant-specific kinase inhibitor with a more potent enzymatic inhibitory activity towards mutant forms of EGFR compared to wild type EGFR. It covalently binds to and irreversibly blocks the catalytic activity of common EGFR mutants (L858R and exon 19 deletions) and certain uncommon EGFR mutants including T790M. In cellular assays comparing EGFR mutant with EGFR wild type cell lines, compound A exhibits potent inhibition of proliferation of mutated cell lines at approximately 35-fold lower concentration than the one observed for inhibition of cells expressing wild type EGFR receptor. Multiple in vivo xenograft studies in mice using different NSCLC models (HCC827 (EGFRDelE746-A750) and H1975 (EGFRL858R/T790M)) confirmed the anti-tumor activity of compound A as a single agent. Tumor regressions were observed in all models. Anti-tumor efficacy was independent of schedule (once daily versus twice daily administration) and was tolerated by the mice at clinically relevant exposure. Compound A is a novel, 3^{rd} generation EGFR mutant-specific TKI, which is currently being investigated in first and second line setting for treatment of patients with EGFR-mutated NSCLC.

Additionally, the 3G-EGFR inhibitors within this invention can be selected from the group consisting of osimertinib (AZD9291), rociletinib (CO-1686), ASP8273, PF-06747775, avitinib (AC0010) and EGF816 and their pharmaceutically acceptable salts. Synthesis and properties of these compounds are also known in the art.

In one aspect the 3G-EGFR inhibitor compound A used in the various embodiments of the invention as described herein is in the form of a hydrochloride salt. Preferably, the hydrochloride salt form of compound A is a crystalline dihydrochloride salt.

To be used in therapy, the 3G-EGFR inhibitor is included into pharmaceutical compositions appropriate to facilitate administration to animals or humans.

Typical pharmaceutical compositions for administering the 3G-EGFR inhibitor of the invention include for example tablets, capsules, suppositories, solutions, e.g. solutions for injection (s.c., i.v., i.m.) and infusion, elixirs, emulsions or dispersible powders. The content of the pharmaceutically active compound(s) may be in the range from 0.1 to 90 wt.-%, preferably 40 to 60 wt.-% of the composition as a whole, e.g. in amounts which are sufficient to achieve the desired dosage range. The single dosages may, if necessary, be given several times a day to deliver the desired total daily dose.

Typical tablets may be obtained, for example, by mixing the active substance(s), optionally in combination, with known excipients, for example inert diluents such as calcium carbonate, calcium phosphate, cellulose or lactose, disintegrants such as corn starch or alginic acid or crospovidon, binders such as starch or gelatine, lubricants such as magnesium stearate or talc and/or agents for delaying release, such as carboxymethyl cellulose, cellulose acetate phthalate, or polyvinyl acetate. The tablets may be prepared by usual processes, such as e.g. by direct compression or roller compaction. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substance(s) may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing the active substance(s) may for example be prepared by mixing the active substance(s) with inert carriers such as lactose or sorbitol and packing them into gelatine capsules.

Typical suppositories may be made for example by mixing the active substance(s) with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The 3G-EGFR inhibitor of this invention is administered by the usual methods, preferably by oral or parenteral route, most preferably by oral route. For oral administration the tablets may contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for oral use is from 1 mg to 2000 mg per day (e.g. for compound A the dosage in the various embodiments of the invention as described herein is from 300 mg to 1200 mg per day; in a more preferred embodiment from 500 mg to 900 mg per day; most preferred is 800 mg per day. The dosage for intravenous use is from 1 mg to 1000 mg per hour, preferably between 5 and 500 mg per hour. All amounts given refer to the free base of compound A and may be proportionally higher if a pharmaceutically acceptable salt or other solid form, e.g. the dihydrochloride salt of compound A, is used. Preferably, the daily dosage is administered once daily (q.d.).

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

### Irreversible (2^{nd} Generation) EGFR TKI

The irreversible (2^{nd} generation) EGFR TKI within the meaning of this invention is afatinib (compound B).

Afatinib (BIBW2992) is a small molecule, potent, selective and irreversible ErbB family blocker. In preclinical models it effectively inhibits signalling from all homo- and heterodimers formed by the ErbB family members EGFR (ErbB1), HER2 (ErbB2), ErbB3 and ErbB4 resulting in tumour growth inhibition and regression of established subcutaneous tumours derived from four human cell-lines known to co-express ErbB receptors. Afatinib is approved as monotherapy to treat patients with advanced or metastatic NSCLC whose tumours have EGFR activating mutations.

The chemical structure of compound B (afatinib) is depicted below.

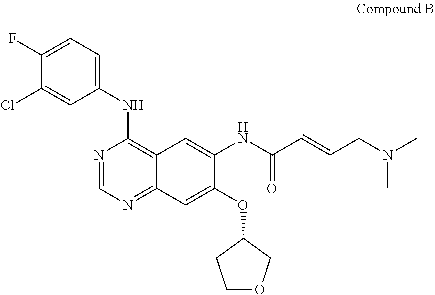 [SMILES: CN(C)C/C=C/C(=O)Nc1cc2c(Nc3ccc(F)c(Cl)c3)ncnc2cc1O[C@H]1CCOC1]

The base form of this compound is described in WO 01/27081 (example compound 1(10)), the dimaleate salt form is described in WO 2005/037824. The use of this molecule for the treatment of oncological diseases is being described in WO 2007/054550, WO 2008/034776 and WO 2011/069962 (all incorporated by reference in their entirety).

The dimaleate salt form of this compound (depicted below) has properties which makes this salt form especially suitable for development as medicament.

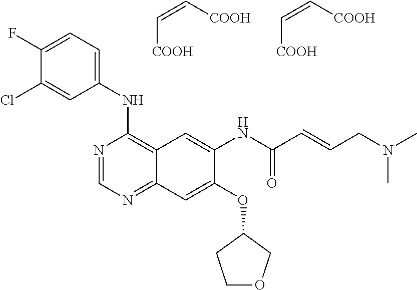 [SMILES: CN(C)C/C=C/C(=O)Nc1cc2c(Nc3ccc(F)c(Cl)c3)ncnc2cc1O[C@H]1CCOC1 ; O=C(O)/C=C\C(=O)O ; O=C(O)/C=C\C(=O)O]

Thus, in one aspect the irreversible (2^{nd} generation) EGFR TKI compound B used in the various embodiments of the invention as described herein is in the form of its dimaleate salt, preferably a crystalline dimaleate salt.

To be used in therapy, the irreversible (2^{nd} generation) EGFR TKI compound B is included into pharmaceutical compositions appropriate to facilitate administration to animals or humans.

Suitable carrier systems (formulations), in particular solid oral formulations, e.g. tablets, including compound B dimaleate are disclosed in WO 2009/147238 (incorporated by reference in its entirety), e.g. tablets containing compound B dimaleate corresponding to 20 mg, 30 mg, 40 mg, 50 mg or 70 mg of compound B (free base). Tablets with a content of compound B dimaleate corresponding to 20 mg, 30 mg, 40 mg and 50 mg of compound B (free base) are commercially available (Giotrif®).

Thus, for compound B the dosage in the various embodiments of the invention as described herein is preferably from 20 mg to 50 mg per day; in a more preferred embodiment from 40 mg to 50 mg per day (all ranges referring to the corresponding amount of the free base of compound B). The daily dosage is preferably administered once daily (q.d.).

For a more detailed description of compound B and its use it is referred to the Summary of Product Characteristics (incorporated by reference in its entirety).

In one embodiment of the invention the irreversible (2^{nd} generation) EGFR TKI is compound B (afatinib)—or a pharmaceutically acceptable salt thereof (preferably the dimaleate salt thereof).

Additionally, the irreversible (2^{nd} generation) EGFR TKI within this invention can also be dacomitinib or a pharmaceutically acceptable salt thereof. Synthesis and properties of this compound are also known in the art.

### Combination Therapy

Within this invention it is to be understood that the combinations, compositions, kits, methods, uses or compounds for use according to this invention may envisage the simultaneous, concurrent, sequential, successive, alternate or separate administration of the active ingredients or components. It will be appreciated that the 3G-EGFR inhibitor and the irreversible (2^{nd} generation) EGFR TKI can be administered formulated either dependently or independently, such as e.g. the 3G-EGFR inhibitor and the irreversible (2^{nd} generation) EGFR TKI may be administered either as part of the same pharmaceutical composition/dosage form or, preferably, in separate pharmaceutical compositions/dosage forms.

In this context, “combination” or “combined” within the meaning of this invention includes, without being limited, a product that results from the mixing or combining of more than one active ingredient and includes both fixed and non-fixed (e.g. free) combinations (including kits) and uses, such as e.g. the simultaneous, concurrent, sequential, successive, alternate or separate use of the components or ingredients. The term “fixed combination” means that the active ingredients are both administered to a patient simultaneously in the form of a single entity or dosage. The term “non-fixed combination” means that the active ingredients are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The administration of the 3G-EGFR inhibitor and the irreversible (2^{nd} generation) EGFR TKI may take place by co-administering the active components or ingredients, such as e.g. by administering them simultaneously or concurrently in one single or in two separate formulations or dosage forms. Alternatively, the administration of the 3G-EGFR inhibitor and the irreversible (2^{nd} generation) EGFR TKI may take place by administering the active components or ingredients sequentially or in alternation, such as e.g. in two separate formulations or dosage forms.

For example, simultaneous administration includes administration at substantially the same time. This form of administration may also be referred to as “concomitant” administration. Concurrent administration includes administering the active agents within the same general time period, for example on the same day(s) but not necessarily at the same time. Alternate administration includes administration of one agent during a time period, for example over the course of a few days or a week, followed by administration of the other agent during a subsequent period of time, for example over the course of a few days or a week, and then repeating the pattern for one or more cycles. Sequential or successive administration includes administration of one agent during a first time period (for example over the course of a few days or a week) using one or more doses, followed by administration of the other agent during a second time period (for example over the course of a few days or a week) using one or more doses. An overlapping schedule may also be employed, which includes administration of the active agents on different days over the treatment period, not necessarily according to a regular sequence. Variations on these general guidelines may also be employed, e.g. according to the agents used and the condition of the subject.

The elements of the combinations of this invention may be administered (whether dependently or independently) by methods customary to the skilled person, e.g. by oral, enterical, parenteral (e.g., intramuscular, intraperitoneal, intravenous, transdermal or subcutaneous injection, or implant), nasal, vaginal, rectal, or topical routes of administration and may be formulated, alone or together, in suitable dosage unit formulations containing conventional non-toxic pharmaceutically acceptable carriers, excipients and/or vehicles appropriate for each route of administration.

Accordingly, in one aspect of the invention, the invention provides a method of treating and/or preventing an oncological or hyperproliferative disease, in particular cancer (such as e.g. the cancer disorders described herein), comprising administering to a patient in need thereof a therapeutically effective amount of a 3G-EGFR inhibitor and a therapeutically effective amount of an irreversible (2^{nd} generation) EGFR TKI (each as described herein), wherein the 3G-EGFR inhibitor is administered simultaneously, concurrently, sequentially, successively, alternately or separately with the irreversible (2^{nd} generation) EGFR TKI.

In another aspect, the invention provides a 3G-EGFR inhibitor as described herein for use in a method of treating and/or preventing an oncological or hyperproliferative disease, in particular cancer, said method comprising administering the 3G-EGFR inhibitor in combination with an irreversible (2^{nd} generation) EGFR TKI as described herein, wherein the 3G-EGFR inhibitor is administered simultaneously, concurrently, sequentially, successively, alternately or separately with the irreversible (2^{nd} generation) EGFR TKI.

In another aspect, the invention provides an irreversible (2^{nd} generation) EGFR TKI as described herein for use in a method of treating and/or preventing an oncological or hyperproliferative disease, in particular cancer, said method comprising administering the irreversible (2^{nd} generation) EGFR TKI in combination with a 3G-EGFR inhibitor as described herein, wherein the irreversible (2^{nd} generation) EGFR TKI is administered simultaneously, concurrently, sequentially, successively, alternately or separately with the 3G-EGFR inhibitor.

In another aspect, the invention provides the use of a 3G-EGFR inhibitor as described herein for preparing a pharmaceutical composition for treating and/or preventing an oncological or hyperproliferative disease, in particular cancer (such as e.g. a cancer disease as described herein), wherein the 3G-EGFR inhibitor is to be used in combination with an irreversible (2^{nd} generation) EGFR TKI as described herein and wherein the 3G-EGFR inhibitor is to be administered simultaneously, concurrently, sequentially, successively, alternately or separately with the irreversible (2^{nd} generation) EGFR TKI.

In another aspect, the invention provides the use of an irreversible (2^{nd} generation) EGFR TKI as described herein for preparing a pharmaceutical composition for treating and/or preventing an oncological or hyperproliferative disease, in particular cancer (such as e.g. a cancer disease as described herein), wherein the irreversible (2^{nd} generation) EGFR TKI is to be used in combination with a 3G-EGFR inhibitor as described herein and wherein the irreversible (2^{nd} generation) EGFR TKI is to be administered simultaneously, concurrently, sequentially, successively, alternately or separately with the 3G-EGFR inhibitor.

In another aspect, the invention provides a kit comprising
a first pharmaceutical composition or dosage form comprising a 3G-EGFR inhibitor as described herein, and, optionally one or more pharmaceutically acceptable carriers, excipients and/or vehicles, and
a second pharmaceutical composition or dosage form comprising an irreversible (2^{nd} generation) EGFR TKI as described herein, and, optionally one or more pharmaceutically acceptable carriers, excipients and/or vehicles,

for use in a method of treating and or/preventing an oncological or hyperproliferative disease, in particular cancer, wherein the first pharmaceutical composition is to be administered simultaneously, concurrently, sequentially, successively, alternately or separately with the second pharmaceutical composition.

In a further embodiment of the invention, the components (i.e. the combination partners) of the combinations, kits, uses, methods and compounds for use according to the invention are administered simultaneously.

In a further embodiment of the invention, the components (i.e. the combination partners) of the combinations, kits, uses, methods and compounds for use according to the invention are administered concurrently.

In a further embodiment of the invention, the components (i.e. the combination partners) of the combinations, kits, uses, methods and compounds for use according to the invention are administered sequentially.

In a further embodiment of the invention, the components (i.e. the combination partners) of the combinations, kits, uses, methods and compounds for use according to the invention are administered successively.

In a further embodiment of the invention, the components (i.e. the combination partners) of the combinations, kits, uses, methods and compounds for use according to the invention are administered alternately.

In a further embodiment of the invention, the components (i.e. the combination partners) of the combinations, kits, uses, methods and compounds for use according to the invention are administered separately.

In a preferred embodiment, the 3G-EGFR inhibitor as described herein is to be administered orally.

In another preferred embodiment, the irreversible (2^{nd} generation) EGFR TKI is to be administered orally.

The “therapeutically effective amount” of the active compound(s) to be administered is the minimum amount necessary to prevent, ameliorate, or treat a disease or disorder.

The combinations of this invention may be administered at therapeutically effective single or divided daily doses. The active components of the combination may be administered in such doses which are therapeutically effective in monotherapy, or in such doses which are lower than the doses used in monotherapy, but when combined result in a desired (jointly) therapeutically effective amount.

In particular embodiments of this invention, the combinations, compositions, kits, methods, uses and compounds for use according to this invention relate to such combinations, compositions, kits, methods, uses and compounds for use in which the 3G-EGFR inhibitor is compound A indicated herein above and the irreversible (2^{nd} generation) EGFR TKI is compound B (afatinib) indicated herein above.

In certain embodiments (embodiments A) of this invention, the combinations, compositions, kits, methods, uses and compounds for use according to this invention refer to such individual pairs of the 3G-EGFR inhibitor and the irreversible (2^{nd} generation) EGFR TKI according to the embodimental entries A1 to A14 (table 1):

TABLE 1

| | | irreversible (2^{nd} generation) |
|---|---|---|
| Embodiment | 3G-EGFR inhibitor | EGFR TKI |
| A1 | Compound A (olmutinib) | Compound B (afatinib) |
| A2 | Compound A (olmutinib) | dacomitinib |
| A3 | osimertinib (AZD9291) | Compound B (afatinib) |
| A4 | osimertinib (AZD9291) | dacomitinib |
| A5 | rociletinib (CO-1686) | Compound B (afatinib) |
| A6 | rociletinib (CO-1686) | dacomitinib |
| A7 | ASP8273 | Compound B (afatinib) |
| A8 | ASP8273 | dacomitinib |
| A9 | PF-06747775 | Compound B (afatinib) |
| A10 | PF-06747775 | dacomitinib |
| A11 | Avitinib (AC0010) | Compound B (afatinib) |
| A12 | Avitinib (AC0010) | dacomitinib |
| A13 | EGF816 | Compound B (afatinib) |
| A14 | EGF816 | dacomitinib |

The combinations, compositions, kits, uses, methods and compounds for use according to the present invention are useful for the treatment and/or prevention of oncological and hyperproliferative disorders.

In certain embodiments the combinations, compositions, kits, uses, methods and compounds for use according to the present invention are useful for the treatment of oncological and hyperproliferative disorders.

In certain embodiments, the hyperproliferative disorder is cancer.

Cancers are classified in two ways: by the type of tissue in which the cancer originates (histological type) and by primary site, or the location in the body, where the cancer first developed. The most common sites in which cancer develops include the skin, lung, breast, prostate, colon and rectum, cervix and uterus as well as the hematological compartment.

The combinations, compositions, kits, uses, methods and compounds for use according to the invention are useful in the treatment of a variety of cancer diseases, including, for example, but not limited to the following:
brain related cancer such as adult brain tumour, childhood brain stem glioma, childhood cerebellar astrocytoma, childhood cerebral astrocytoma/malignant glioma, childhood ependymoma, childhood medulloblastoma, childhood supratentorial primitive neuroectodermal tumours, childhood visual pathway and hypothalamic glioma and other childhood brain tumours;
breast cancer;
digestive/gastrointestinal related cancer such as anal cancer, extrahepatic bile duct cancer, gastrointestinal carcinoid tumour, cholangiocarcinoma, colon cancer, esophageal cancer, gallbladder cancer, adult primary liver cancer (hepatocellular carcinoma, hepatoblastoma) childhood liver cancer, pancreatic cancer, rectal cancer, small intestine cancer and stomach (gastric) cancer;
endocrine related cancer such as adrenocortical carcinoma, gastrointestinal carcinoid tumour, islet cell carcinoma (endocrine pancreas), parathyroid cancer, pheochromocytoma, pituitary tumour and thyroid cancer;
eye related cancer such as intraocular melanoma, and retinoblastoma;
genitourinary related cancer such as bladder cancer, kidney (renal cell) cancer, penile cancer, prostate cancer, transitional cell renal pelvis and ureter cancer, testicular cancer, urethral cancer, Wilms' tumour and other childhood kidney tumours;
germ cell related cancer such as childhood extracranial germ cell tumour, extragonadal germ cell tumour, ovarian germ cell tumour and testicular cancer;
gynecologic cancer such as cervical cancer, endometrial cancer, gestational trophoblastic tumour, ovarian epithelial cancer, ovarian germ cell tumour, ovarian low malignant potential tumour, uterine sarcoma, vaginal cancer and vulvar cancer;
head and neck related cancer such as hypopharyngeal cancer, laryngeal cancer, lip and oral cavity cancer, metastatic squamous neck cancer with occult primary, nasopharyngeal cancer, oropharyngeal cancer, paranasal sinus and nasal cavity cancer (e.g. sinonasal squamouns cell carcinoma), parathyroid cancer and salivary gland cancer;
hematologic/blood related cancer such as leukemias, such as adult acute lymphoblastic leukemia, childhood acute lymphoblastic leukemia, adult acute myeloid leukemia, childhood acute myeloid leukemia, chronic lymphocytic leukemia, chronic myelogenous leukemia and hairy cell leukemia; and lymphomas, such as AIDS-related lymphoma, cutaneous T-cell lymphoma, adult Hodgkin's lymphoma, childhood Hodgkin's lymphoma, Hodgkin's lymphoma during pregnancy, mycosis fungoides, adult non-Hodgkin's lymphoma, childhood non-Hodgkin's lymphoma, non-Hodgkin's lymphoma during pregnancy, primary central nervous system lymphoma, Sezary syndrome, cutaneous T-cell lymphoma and Waldenström's macroglobulinemia and other hematologic/blood related cancer such as chronic myeloproliferative disorders, multiple myeloma/plasma cell neoplasm, myelodysplastic syndromes and myelodysplastic/myeloproliferative diseases;
musculoskeletal related cancer such as Ewing's family of tumours, osteosarcoma, malignant fibrous histiocytoma of bone, childhood rhabdomyosarcoma, adult soft tissue sarcoma, childhood soft tissue sarcoma and uterine sarcoma; hemangiosarcomas and angiosarcoma;
neurologic related cancer such as adult brain tumour, childhood brain tumour, brain stem glioma, cerebellar astrocytoma, cerebral astrocytoma/malignant glioma, ependmoma, medulloblastoma, supratentorial primitive neuroectodermal tumours, visual pathway and hypothalamic glioma and other brain tumours such as neuroblastoma, pituitary tumour and primary central nervous system lymphoma;
respiratory/thoracic related cancer such as non-small cell lung cancer (NSCLC), small cell lung cancer (SCLC), squamous cell carcinoma (SCC) of the lung, malignant mesothelioma, thymoma and thymic carcinoma;
skin related cancer such as cutaneous T-cell lymphoma, Kaposi's sarcoma, melanoma, Merkel cell carcinoma and skin cancer;
small blue round cell tumours.

In a further embodiment, the combinations, compositions, kits, uses, methods and compounds for use of the invention are beneficial in the treatment of cancers of the hematopoietic system including leukemias, lymphomas and myelomas, cancers of the gastrointestinal tract including esophageal, gastric, colorectal, pancreatic, liver and gall bladder and bile duct cancer; kidney, prostate and bladder cancer; gynecological cancers including breast, ovarian, cervical and endometrial cancer; skin and head and neck cancers including malignant melanomas; pediatric cancers like Wilms' tumour, neuroblastoma and Ewing'sarcoma; brain cancers like glioblastoma; sarcomas like osteosarcoma, soft tissue sarcoma, rhabdomyosarcoma, hemangiosarcoma; lung cancer including non-small cell lung cancer, mesothelioma and thyroid cancer.

In a further embodiment of the invention, the combinations, compositions, kits, uses, methods and compounds for use according to the invention are used to treat non-small cell lung cancer (NSCLC) (including for example locally advanced or metastatic NSCLC (stage IIIB/IV), NSCLC adenocarcinoma, NSCLC with squamous histology, NSCLC with non-squamous histology).

In a further embodiment of the invention, the combinations, compositions, kits, uses, methods and compounds for use according to the invention are used in the treatment of non-small cell lung cancer (NSCLC), in particular NSCLC adenocarcinoma.

In a further embodiment of the invention, the combinations, compositions, kits, uses, methods and compounds for use according to the invention are used in the treatment of non-small cell lung cancer (NSCLC) characterized by aberrant activation, or amplification, or mutations of EGFR.

In a further embodiment of the invention, the combinations, compositions, kits, uses, methods and compounds for use according to the invention are used in the treatment of a cancer harboring one or more EGFR mutation.

In a further embodiment of the invention, the combinations, compositions, kits, uses, methods and compounds for use according to the invention are used in the treatment of a cancer harboring an EGFR exon 20 insertion or an EGFR exon 19 deletion (Del19) or an EGFR L858R mutation or an EGFR T790M mutation, or any combination thereof.

In a further embodiment of the invention, the combinations, compositions, kits, uses, methods and compounds for use according to the invention are used in the treatment of a cancer harboring one or more EGFR mutations wherein at least one EGFR mutation is selected from Del19 (deletion in exon 19), L858R and T790M.

In a further embodiment of the invention, the combinations, compositions, kits, uses, methods and compounds for use according to the invention are used in the treatment of a cancer harboring the EGFR mutation Del19.

In a further embodiment of the invention, the combinations, compositions, kits, uses, methods and compounds for use according to the invention are used in the treatment of a cancer harboring the EGFR mutation L858R.

In a further embodiment of the invention, the combinations, compositions, kits, uses, methods and compounds for use according to the invention are used in the treatment of a cancer harboring the EGFR mutation T790M.

In a further embodiment of the invention, the combinations, compositions, kits, uses, methods and compounds for use according to the invention are used in the treatment of a cancer harboring at least two EGFR mutations selected from the group consisting of Del19/T790M and L858R/T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring an EGFR exon 20 insertion or an EGFR exon 19 deletion (Del19) or an EGFR L858R mutation or an EGFR T790M mutation, or any combination thereof.

In a further embodiment of the invention, the combinations, compositions, kits, uses, methods and compounds for use according to the invention are used in the treatment of non-small cell lung cancer (NSCLC), in particular NSCLC adenocarcinoma, harboring one or more EGFR mutations wherein at least one EGFR mutation is selected from Del19 (deletion in exon 19), L858R and T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring at least two EGFR mutations selected from the group consisting of Del19/T790M and L858R/T790M.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation Del19.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation L858R.

In a further embodiment of the invention, the combinations, compositions, kits, uses, methods and compounds for use according to the invention are used in the treatment of non-small cell lung cancer (NSCLC), in particular NSCLC adenocarcinoma, harboring the EGFR mutation T790M.

The therapeutic applicability of the combination therapy according to this invention may include first line, second line, third line or further lines of treatment of patients. The cancer may be metastatic, recurrent, relapsed, resistant or refractory to one or more anti-cancer treatments. Thus, the patients may be treatment naïve, or may have received one or more previous anti-cancer therapies, which have not completely cured the disease.

Patients with relapse and/or with resistance to one or more anti-cancer agents (e.g. the single components of the combination, or standard chemotherapeutics) are also amenable for combined treatment according to this invention, e.g. for second or third line treatment cycles (optionally in further combination with one or more other anti-cancer agents), e.g. as add-on combination or as replacement treatment.

Accordingly, some of the disclosed combination therapies of this invention are effective at treating subjects whose cancer has relapsed, or whose cancer has become drug resistant or multi-drug resistant, or whose cancer has failed one, two or more lines of mono- or combination therapy with one or more anti-cancer agents (e.g. the single components of the combination, or standard chemotherapeutics).

A cancer which initially responded to an anti-cancer drug can relapse and it becomes resistant to the anti-cancer drug when the anti-cancer drug is no longer effective in treating the subject with the cancer, e.g. despite the administration of increased dosages of the anti-cancer drug. Cancers that have developed resistance to two or more anti-cancer drugs are said to be multi-drug resistant.

Accordingly, in some methods of combination treatment of this invention, treatment with a combination according to this invention administered secondly or thirdly is begun if the patient has resistance or develops resistance to one or more agents administered initially or previously. The patient may receive only a single course of treatment with each agent or multiple courses with one, two or more agents.

In certain instances, combination therapy according to this invention may hence include initial or add-on combination, replacement or maintenance treatment.

In a further embodiment of the invention, the combinations, compositions, kits, uses, methods and compounds for use according to the invention are used in the treatment of cancers/cancer patients (suffering from cancers as described herein, in particular suffering from NSCLC as described herein) which are treatment naïve, i.e. their cancer disease has not been treated previously. In further embodiments the cancers/cancer patients (suffering from cancers as described herein, in particular suffering from NSCLC as described herein) have been previously treated with 1^{st} generation EGFR TKIs selected from erlotinib and gefitinib. In further embodiments the cancers/cancer patients (suffering from cancers as described herein, in particular suffering from NSCLC as described herein) have been previously treated with 2^{nd} generation EGFR TKIs selected from afatinib and dacomitinib.

The present invention is not to be limited in scope by the specific embodiments described herein. Various modifications of the invention in addition to those described herein may become apparent to those skilled in the art from the present disclosure. Such modifications are intended to fall within the scope of the appended claims.

All patent applications cited herein are hereby incorporated by reference in their entireties.

### REFERENCE

1. Mok T S, Wu Y L, Thongprasert S et al. Gefitinib or carboplatin-paclitaxel in pulmonary adenocarcinoma. N Engl J Med 2009; 361: 947-957.
2. Zhou C, Wu Y L, Chen G et al. Erlotinib versus chemotherapy as first-line treatment for patients with advanced EGFR mutation-positive non-small-cell lung cancer (OPTIMAL, CTONG-0802): a multicentre, open-label, randomised, phase 3 study. Lancet Oncol 2011; 12: 735-742.
3. Paez J G, Janne P A, Lee J C et al. EGFR mutations in lung cancer: correlation with clinical response to gefitinib therapy. Science 2004; 304: 1497-1500.
4. Lynch T J, Bell D W, Sordella R et al. Activating mutations in the epidermal growth factor receptor underlying responsiveness of non-small-cell lung cancer to gefitinib. N Engl J Med 2004; 350: 2129-2139.
5. Pao W, Miller V, Zakowski M et al. EGF receptor gene mutations are common in lung cancers from “never smokers” and are associated with sensitivity of tumors to gefitinib and erlotinib. Proc Natl Acad Sci USA 2004; 101: 13306-13311.
6. Rosell R, Carcereny E, Gervais R et al. Erlotinib versus standard chemotherapy as first-line treatment for European patients with advanced EGFR mutation-positive non-small-cell lung cancer (EURTAC): a multicentre, open-label, randomised phase 3 trial. Lancet Oncol 2012; 13: 239-246.
7. Fukuoka M, Wu Y L, Thongprasert S et al. Biomarker analyses and final overall survival results from a phase III, randomized, open-label, first-line study of gefitinib versus carboplatin/paclitaxel in clinically selected patients with advanced non-small-cell lung cancer in Asia (IPASS). J Clin Oncol 2011; 29: 2866-2874.
8. Kobayashi S, Boggon T J, Dayaram T et al. EGFR mutation and resistance of non-small-cell lung cancer to gefitinib. N Engl J Med 2005; 352: 786-792.
9. Morita S, Okamoto I, Kobayashi K et al. Combined survival analysis of prospective clinical trials of gefitinib for non-small cell lung cancer with EGFR mutations. Clin Cancer Res 2009; 15: 4493-4498.
10. Pao W, Miller V A, Politi K A et al. Acquired resistance of lung adenocarcinomas to gefitinib or erlotinib is associated with a second mutation in the EGFR kinase domain. PLoS Med 2005; 2: e73.
11. Riely G J, Pao W, Pham D et al. Clinical course of patients with non-small cell lung cancer and epidermal growth factor receptor exon 19 and exon 21 mutations treated with gefitinib or erlotinib. Clin Cancer Res 2006; 12: 839-844.
12. Zhou W, Ercan D, Chen L et al. Novel mutant-selective EGFR kinase inhibitors against EGFR T790M. Nature 2009; 462: 1070-1074.
13. Cross D A, Ashton S E, Ghiorghiu S et al. AZD9291, an irreversible EGFR TKI, overcomes T790M-mediated resistance to EGFR inhibitors in lung cancer. Cancer Discov 2014; 4: 1046-1061.
14. Planchard D. O10.4AZD9291 a novel EGFR-TKI that overcomes T790M-mediated resistance in NSCLC. Ann Oncol 2015; 26 Suppl 2: ii14.
15. Soria J C, Sequist L V, Goldman J et al. O10.3Rociletinib: an oral, irreversible, highly selective small molecule inhibitor of mutant EGFR including T790M. Ann Oncol 2015; 26 Suppl 2: ii14.
16. Steuer C E, Khuri F R, Ramalingam S S. The next generation of epidermal growth factor receptor tyrosine kinase inhibitors in the treatment of lung cancer. Cancer 2015; 121: E1-6.
17. Jiang T, Zhou C. Clinical activity of the mutant-selective EGFR inhibitor AZD9291 in patients with EGFR inhibitor-resistant non-small cell lung cancer. Transl Lung Cancer Res 2014; 3: 370-372.
18. Finlay M R, Anderton M, Ashton S et al. Discovery of a potent and selective EGFR inhibitor (AZD9291) of both sensitizing and T790M resistance mutations that spares the wild type form of the receptor. J Med Chem 2014; 57: 8249-8267.
19. Thress K S, Paweletz C P, Felip E, Cho B C, Stetson D, Dougherty B, Lai Z, Markovets A, Vivancos A, Kuang Y, Ercan D, Matthews S E, Cantarini M, Barrett J C, Janne P A, Oxnard G R. Acquired EGFR C797S mutation mediates resistance to AZD9291 in non-small cell lung cancer harboring EGFR T790M. Nature Med 2015. 221(6):560-562.
20. Ercan D, Choi H G, Yun C H, Capelletti M, Xie T, Eck M J, Gray N S, Janne P A. EGFR mutations and resistance to irreversible pyrimidine-based EGFR inhibitors. Clin Cancer Res 21 (17), 3913-3923 (2015)
21. Niederst M J, Hu H, Mulvey H E, Lockerman E L, Garcia A R, Piotrowska Z, Sequist L V, Engelman J A. The allelic context of the C797S mutation acquired upon treatment with third-generation EGFR inhibitors impacts sensitivity to subsequent treatment strategies. Clin Cancer Res 21 (17), 3924-3933 (2015)

### EXAMPLE 1: PHASE IB STUDY OF BI 1482694 (COMPOUND A) IN COMBINATION WITH AFATINIB (COMPOUND B) IN PATIENTS WITH EGFR-MUTANT LOCALLY ADVANCED OR METASTATIC NON-SMALL CELL LUNG CANCER

### Introduction

The study proposed here investigates the effect of BI 1482694 (compound A) in combination with afatinib (compound B) in patients with EGFR-mutant locally advanced or metastatic non-small cell lung cancer (NSCLC).

### Objectives

Dose finding: To determine the maximum tolerated dose (MTD) and the anti-tumour activity of BI 1482694 (compound A) in combination with afatinib (compound B) in patients with EGFR-mutant locally advanced/metastatic NSCLC previously treated or not with EGFR TKI.

Expansion: To assess anti-tumour activity of BI 1482694 (compound A) in combination with afatinib (compound B) in EGFR TKI naïve patients.

### Methodology

Prospective, open-label, non-randomised, phase I study with a dose finding part followed by an expansion part.

### Diagnosis

Patients with locally advanced (stage IIIb) or metastatic (stage IV) EGFR mutant NSCLC.

### Main Criteria for Inclusion

Pathologically confirmed diagnosis of non-squamous carcinoma of the lung (NSCLC);
Locally advanced (stage IIIb) or metastatic (stage IV) EGFR-mutant NSCLC;
At least one documented EGFR mutation known to be associated with EGFR TKI sensitivity, e.g. Del 19, L858R, L861Q, G719X, S768I;
For patients pre-treated with EGFR TKI: Radiologically confirmed progression or recurrence of disease during or following the most recent prior treatment;
Expansion part only: At least one target lesion (excluding the brain), that can be accurately measured per RECIST version 1.1. In patients who have only one target lesion, and a biopsy of this lesion is required, the baseline imaging must be performed at least 2 weeks after the biopsy.
Previous treatment:
   Dose finding part: treatment naïve or pre-treated with EGFR TKI (1^{st} and/or 2^{nd} and/or 3^{rd} generation). One prior line of chemotherapy and one prior line of immunotherapy for stage IIIb/IV NSCLC are permitted;
   Expansion part: no previous EGFR TKI treatment (EGFR TKI naïve), one prior line of chemotherapy is allowed;
Expansion part only: at least one target lesion (excluding the brain), that can be accurately measured per Response Evaluation Criteria in Solid Tumours (RECIST) version 1.1.
Eastern Cooperative Oncology Group (ECOG) performance score of 0 or 1
Adequate organ function defined as all of the following:
   Absolute neutrophil count (ANC)≥1.5×10⁹/L; hemoglobin≥9.0 g/dL; platelets≥100×10⁹/L, without the use of hematopoietic growth factors within 4 weeks of start of study medication.
   Total bilirubin≤1.5 times the upper limit of normal (ULN), or ≤4×ULN for patients who are known to have Gilbert's syndrome.
   Creatinine≤1.5×ULN. If creatinine is ≥1.5×ULN, patient is eligible if concurrent creatinine clearance≥50 ml/min (measured or calculated by Cockcroft-Gault formula).
   Aspartate transaminase (AST) and alanine transaminase (ALT)≤3×ULN if no demonstrable liver metastases, or otherwise ≤5×ULN
Recovered from any previous therapy-related toxicity to ≤CTCAE Grade 1 at first administration of study drug (except for alopecia and stable sensory neuropathy which must be ≤CTCAE Grade 2)

### Main Criteria for Exclusion

More than four lines of prior therapy for stage IIIb/IV. Expansion cohort: any prior therapy for stage IIIb/IV
   a. Radiotherapy alone is not counted as a line of therapy.
   b. Radiosensitisers and/or intrapleural administration of anti-cancer agents are not counted as a line of therapy.
   c. Prior neoadjuvant/adjuvant systemic therapy is not counted as a line of therapy if therapy was completed at least 6 months prior to disease relapse.
Previous treatment with:
   a. Previous treatment with EGFR-TKI, within 8 days or 5 half-lives, whichever is longer, prior to first dose of trial treatment. Treatment with EGFR TKIs during screening is allowed as long as the washout period of 8 days or 5 half-lives is guaranteed. Expansion cohort: any previous treatment with an EGFR TKI.
   b. Previous experimental anticancer therapy within 4 weeks; previous chemotherapy or anticancer immunotherapy (except mAbs) or anticancer hormonal treatment within 2 weeks of the first administration of study drug.
   c. Prior anti-cancer monoclonal antibody (mAb) within 4 weeks prior to the first dose of trial treatment.
   d. Radiotherapy within 4 weeks prior to first dose of trial treatment except as follows:
      Palliative radiotherapy to regions other than the chest is allowed up to 2 weeks prior to first dose of trial treatment.
      Single dose palliative radiotherapy for symptomatic metastasis within 2 weeks prior to first dose of trial treatment may be allowed but must be discussed with the sponsor.
   e. Major surgery within 4 weeks prior to first dose of trial treatment or scheduled during the projected course of the study.
Known history of hypersensitivity to BI 1482694 (compound A) or any of its excipients or drugs with a chemical structure similar to BI 1482694.
Known history of hypersensitivity to afatinib (compound B) or any of its excipients or drugs with a similar chemical structure.
History or presence of cardiovascular abnormalities such as uncontrolled hypertension, congestive heart failure NYHA classification of ≥3, unstable angina or poorly controlled arrhythmia which are considered as clinically relevant by the Investigator. Myocardial infarction within 6 months prior to first dose of trial treatment.
Previous or concomitant malignancies at other sites, except effectively treated:
   non-melanoma skin cancers
   carcinoma in situ of the cervix
   ductal carcinoma in situ
   other malignancy that has been in remission for more than 3 years and is considered to be cured.
Known pre-existing interstitial lung disease or radiation pneumonitis
Any history or presence of uncontrolled gastrointestinal disorders that could affect the intake and/or absorption of the study drug (e.g. nausea, vomiting, Crohn's disease, ulcerative colitis, chronic diarrhoea, malabsorption) in the opinion of the Investigator
Known active hepatitis B infection (defined as presence of HepB sAg and/or Hep B DNA), active hepatitis C infection (defined as presence of Hep C RNA) and/or known HIV carrier.
Active infectious disease which puts the patient at increased risk in the opinion of the Investigator
Left ventricular ejection fraction (LVEF)<50%.
Leptomeningeal carcinomatosis
Presence or history of uncontrolled or symptomatic brain or subdural metastases, unless considered stable by the investigator and local therapy was completed. Use of corticosteroids is allowed if the dose was stable for at least 4 weeks. Inclusion of patients with newly identified brain metastasis/es at screening will be allowed if patients are asymptomatic.

### Dose

### Dose Finding Part:

Starting dose of BI 1482694 (compound A) is 600 mg once daily in combination with afatinib (compound B) at a starting dose of 20 mg once daily.

Expansion phase:

MTD of BI 1482694 (compound A) in combination with afatinib (compound B) as determined in the dose finding part.

### Mode of Administration

BI 1482694 (compound A): p.o. once daily
Afatinib (compound B): p.o. once daily

### Duration of Treatment

BI 1482694 (compound A) in combination with afatinib (compound B) will be given until disease progression, unacceptable treatment-related adverse events or other reasons requiring treatment discontinuation.

### Endpoints

Primary endpoints for the dose finding part:
Maximum tolerated dose (MTD).
Number of patients with dose limiting toxicities (DLTs) during the MTD evaluation period (first 28-day course)

### Primary Endpoint for the Expansion Part:

Progression-free survival (PFS)

### Secondary Endpoints for Both the Dose Finding and the Expansion Part:

Objective response (CR, PR per RECIST version 1.1).
Disease control (CR/PR/SD per RECIST version 1.1).
Duration of objective response
Duration of disease control
Tumour shrinkage

1. A method of treating and/or preventing an oncological or hyperproliferative disease, in particular cancer, comprising administering to a patient in need thereof a therapeutically effective amount of Compound A 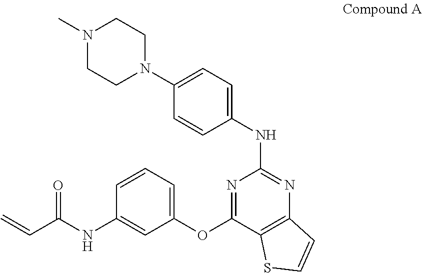 [SMILES: C=CC(=O)Nc1cccc(Oc2nc(Nc3ccc(N4CCN(C)CC4)cc3)nc3ccsc23)c1]
—or of a pharmaceutically acceptable salt thereof—
and a therapeutically effective amount of Compound B 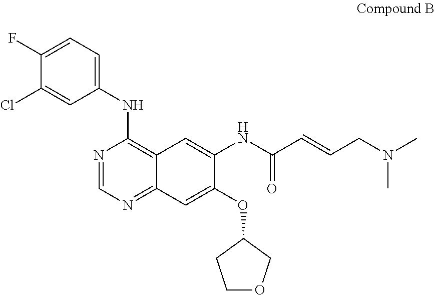 [SMILES: CN(C)C/C=C/C(=O)Nc1cc2c(Nc3ccc(F)c(Cl)c3)ncnc2cc1O[C@H]1CCOC1]
—or of a pharmaceutically acceptable salt thereof.

2. The method according to claim 1, wherein Compound A—or a pharmaceutically acceptable salt thereof—is administered simultaneously, concurrently, sequentially, successively, alternately or separately with Compound B—or a pharmaceutically acceptable salt thereof.

3. (canceled)

4. (canceled)

5. (canceled)

6. (canceled)

7. (canceled)

8. (canceled)

9. (canceled)

10. (canceled)

11. A pharmaceutical composition comprising:
Compound A 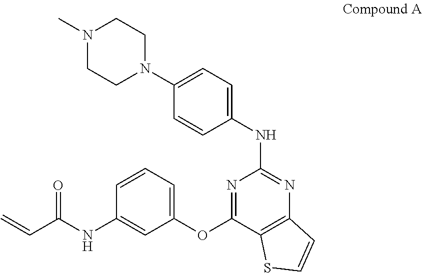 [SMILES: C=CC(=O)Nc1cccc(Oc2nc(Nc3ccc(N4CCN(C)CC4)cc3)nc3ccsc23)c1]
—or a pharmaceutically acceptable salt thereof—and
Compound B 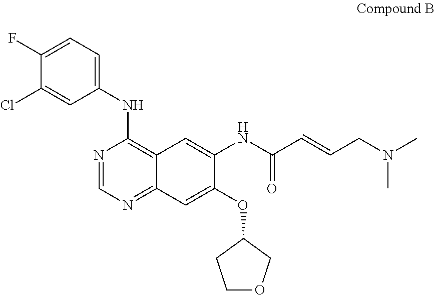 [SMILES: CN(C)C/C=C/C(=O)Nc1cc2c(Nc3ccc(F)c(Cl)c3)ncnc2cc1O[C@H]1CCOC1]
—or a pharmaceutically acceptable salt thereof, and,
optionally, one or more pharmaceutically acceptable carriers, excipients and/or vehicles.

12. The pharmaceutical composition according to claim 11 for use in a method of treating and/or preventing an oncological or hyperproliferative disease, in particular cancer.

13. A kit comprising:
a first pharmaceutical composition comprising Compound A 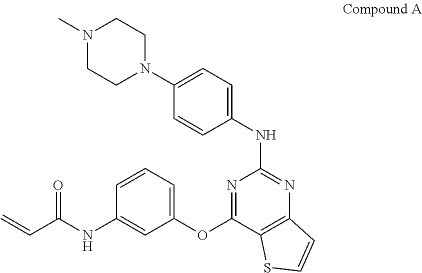 [SMILES: C=CC(=O)Nc1cccc(Oc2nc(Nc3ccc(N4CCN(C)CC4)cc3)nc3ccsc23)c1]
—or a pharmaceutically acceptable salt thereof—and, optionally, one or more pharmaceutically acceptable carriers, excipients and/or vehicles; and
a second pharmaceutical composition comprising Compound B 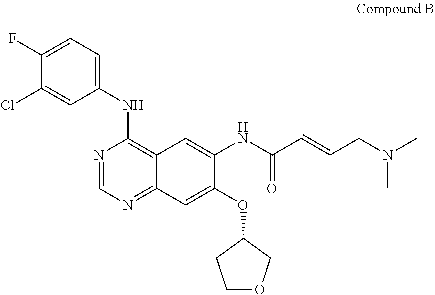 [SMILES: CN(C)C/C=C/C(=O)Nc1cc2c(Nc3ccc(F)c(Cl)c3)ncnc2cc1O[C@H]1CCOC1]
—or a pharmaceutically acceptable salt thereof—and, optionally, one or more pharmaceutically acceptable carriers, excipients and/or vehicles.

14. The kit according to claim 13, for use in a method of treating and/or preventing an oncological or hyperproliferative disease, in particular cancer.

15. The kit according to claim 14, wherein the first pharmaceutical composition is to be administered simultaneously, concurrently, sequentially, successively, alternately or separately with the second pharmaceutical composition.

16. The kit according to any claim 13, further comprising
a package insert comprising printed instructions for simultaneous, concurrent, sequential, successive, alternate or separate use in the treatment and/or prevention of an oncological or hyperproliferative disease, in particular cancer, in a patient in need thereof.

17. The method according to claim 1, wherein Compound A is in the form of a hydrochloride salt, preferably a crystalline dihydrochloride salt.

18. The method according to claim 1, wherein Compound B is in the form of a dimaleate salt, preferably a crystalline dimaleate salt.

19. The method according to claim 1, wherein the oncological disease to be treated is cancer harboring one or more EGFR mutation.

20. The method according to claim 1, wherein the oncological disease to be treated is cancer harboring an EGFR exon 20 insertion or an EGFR exon 19 deletion (Del19) or an EGFR L858R mutation or an EGFR T790M mutation, or any combination thereof.

21. The method according to claim 19, wherein at least one EGFR mutation is selected from Del19 (deletion in exon 19), L858R and T790M.

22. The method according to claim 21, wherein the at least one EGFR mutation is Del19.

23. The method according to claim 21, wherein the at least one EGFR mutation is L858R.

24. The method according to claim 21, wherein the at least one EGFR mutation is T790M.

25. The method according to claim 21, wherein the cancer harbors at least two EGFR mutations selected from the group consisting of Del19/T790M and L858R/T790M.

26. The method according to claim 19, wherein the cancer is non-small cell lung cancer (NSCLC), preferably non-small cell lung cancer adenocarcinoma.
